(19) United States
(12) Patent Application Publication
(10) Pub. No.: US 2004/0241786 A1
(43) Pub. Date: Dec. 2, 2004

(54) Single tube screen

(76) Inventor: Gary W Procop, Twinburg, OH (US)

Correspondence Address: TAROLLI, SUNDHEIM, COVELL & TUMMINO L.L.P., 526 SUPERIOR AVENUE, SUITE 1111, CLEVEVLAND, OH, 44114, US

(21) Appl. No.: 10/495,027

(22) PCT Filed: Nov. 22, 2002
(86) PCT No.: PCT/US02/37501

(30) Foreign Application Priority Data
Nov. 26, 2001 (US) 60333400

(51) Int. Cl. C12Q 1/04

(52) U.S. Cl. 435/34

(57) ABSTRACT

An apparatus (10) for the detection of a microorganism has a chamber (22), a first culture medium disposed (24) within the chamber (22), a second culture medium (26) disposed within the chamber (22), and a barrier (28) that substantially separates the first culture medium (24) and the second culture medium (26) within the chamber (22). The barrier (28) is a semi-solid, hydrophobic material.

### FIELD OF THE INVENTION

The present invention relates to an apparatus and method for the cultivation and detection of a microorganism and particularly relates to an apparatus and method for screening bacterial enteric pathogens.

### BACKGROUND OF THE INVENTION

Stool cultures are used to identify bacteria suspected of infecting a patient's digestive tract. The stool cultures are obtained by placing a sample of the patient's feces on a culture medium that provides nutrients for certain bacteria to grow and reproduce. The medium is usually a thick gel-like substance. The culture is typically performed on a round culture plate that is incubated at the proper temperature for growth of the bacteria.

Colonies of bacteria that grow in the medium are isolated. Isolates of bacteria that are suspected of being pathogens are then screened using a differential culture medium. The differential culture medium provides a preliminary characterization of whether the isolated bacteria is a pathogen.

Two common bacteria pathogens that can cause infection of the digestive tract and can be present in stool samples are Salmonella and Shigella. Bacteria isolated from stool cultures suspected of being Salmonella or Shigella are typically screened using a differential culture medium, such as triple sugar iron agar slant, lysine iron agar slant, and urea media. These media include pH indicators that can change color depending on whether the isolate of bacteria placed in the media undergoes an alkaline or acidic reaction. These differential culture media are provided in separate test tubes so that the pH of one medium does not affect the pH of another medium.

### SUMMARY OF THE INVENTION

One aspect of the present invention relates to an apparatus for the detection of a microorganism. The apparatus comprises a chamber, a first culture medium disposed within the chamber, a second culture medium disposed within the chamber, and a barrier substantially separating the first culture medium and the second culture medium within the chamber. The barrier comprises a semi-solid, hydrophobic material.

Another aspect of the present relates to a method of detecting the microorganism. In the method, an apparatus is provided that includes a chamber, a first culture medium disposed within the chamber, a second culture medium disposed within the chamber, and a barrier. The barrier substantially separates the first culture medium and the second culture medium within the chamber. The barrier comprises a semi-solid, hydrophobic material. The first culture medium and the second culture medium are inoculated with a microorganism. The inoculated first culture medium and the second culture medium are incubated. The incubated first culture medium and second culture medium are examined.

### BRIEF DESCRIPTION OF DRAWINGS

The foregoing and other features of the present invention will become apparent to one skilled in the art upon consideration of the following description of the invention and the accompanying drawings in which:

FIG. 1 is a schematic illustration of an apparatus in accordance with one embodiment of the present invention;

FIG. 2 is a schematic illustration of an apparatus in accordance with another embodiment of the present invention;

FIG. 3 is a schematic illustration of an apparatus in accordance with yet another embodiment of the present invention;

FIG. 4 is a schematic block diagram illustrating a method of producing the apparatus of FIG. 1;

FIG. 5 is a schematic block diagram illustrating a method of using the apparatus of FIG. 1;

FIG. 6 is a flow chart showing a process path for identification of bacteria in accordance with the present invention;

FIG. 7 is a photograph of a single tube screen in accordance with the present invention that has been inoculated with Salmonella and incubated;

FIG. 8 is a photograph of a single tube screen in accordance with the present invention that has been inoculated with Shigella and incubated; and

FIG. 9 is a photograph of a single tube screen in accordance with the present invention that has been inoculated with Proteus and incubated.

### DESCRIPTION OF THE EMBODIMENTS

The present invention relates to an apparatus for the cultivation and detection of a microorganism. As used herein the term “microorganism” includes all microscopic living organisms and cells, including without limitation a virus, bacterium, protozoan, fungus, intracellular parasite, some helminths as well as microscopic forms of eukaryotic cells, for example, single cells (cultured or derived directly from a tissue or organ) or small clumps of cells.

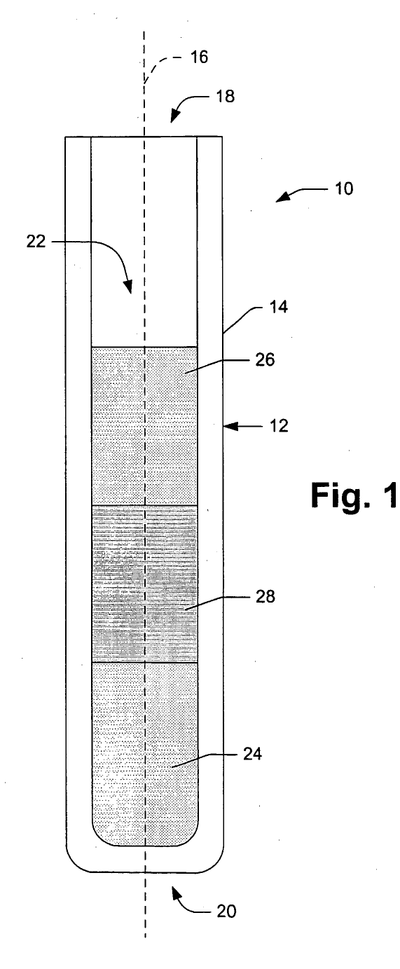

Referring to FIG. 1, which is a schematic illustration of the apparatus 10 in accordance with an aspect of the present invention, the apparatus 10 includes a receptacle 12 with a tubular wall 14. The tubular wall 14 extends along a longitudinal axis 16 between a first open end 18 and a second closed end 20. The tubular wall 14 defines a longitudinally extending chamber 22 that has a volumetric capacity for liquid retention.

The wall 14 of the receptacle 12 is formed from a material that is capable of retaining an aqueous medium. The material can be at least partially transparent so that substances within the chamber can be examined visually or photometrically through the wall 14 of the receptacle 12. Examples of materials that can be used are glass and transparent plastics.

An example of one receptacle that can be used in accordance with present invention is a conventional glass test tube. Other receptacles well known in the art for retention of an aqueous medium, such as a cuvette and a flask, can also be used.

The apparatus 10 further includes two culture media, 24 and 26, and a hydrophobic barrier 28. The two culture media, 24 and 26, and hydrophobic barrier 28 are contained within the chamber 22. The culture media, 24 and 26, can comprise any substance on which a microorganism can grow.

In accordance with one aspect of the present invention, at least one of the two culture media, 24 and 26, is a differential culture medium. By “differential culture medium” it is meant a culture medium that potentially allows one or more different types of microorganisms to grow, and which contains dyes and/or other components upon which the different microrganisms act in various ways to produce a variety of end products or effects, such as variation in color, that can be detected to differentiate the microorganisms.

The culture media used in the present invention are preferably water-based and include one or more ingredients typically used in a culture medium for cultivating microorganisms. Examples of ingredients typically used in a culture medium include sugars, such as dextrose, sucrose, and fructose, lipids, emulsifiers, buffers, extracts, such as eucaryotic tissue, peptones, and reducing agents. The culture media can also include an indicator substance that produces a detectable signal in the presence of a growing microorganism. An example of an indicator substance is a pH indicator (i.e., acid-base indicators) that detects a change in hydrogen ion concentration during growth of a microorganism in the culture medium. Examples of pH indicators are brom-cresol purple and phenol red.

Optionally, at least one of the culture media can include a solidifying agent that acts to at least partially solidify the culture medium. Such solidifying agents are known to those skilled in the art, and can include any water-absorbing material that becomes a gel upon addition of an aqueous liquid. An example of such a solidifying agent is agar.

The hydrophobic barrier 28 comprises a semi-solid or viscous liquid hydrophobic material that is incapable of dissolving in either of the two culture media. One example of a semi-solid hydrophobic material incapable of dissolving in the two culture media is petrolatum (i.e., petroleum jelly). Petrolatum comprises hydrocarbons that are derived by the distillation of paraffin based petroleum fractions. Petrolatum is commercially available under the trade name VASELINE.

The two culture media, 24 and 26, and hydrophobic barrier 28 are axially aligned within the chamber 22 of the receptacle so that the hydrophobic barrier substantially separates the two culture media 24 and 26. Separation of the two culture media 24 and 26, by the hydrophobic barrier 28 is advantageous because it allows for two different pH sensitive culture media to be used in the same chamber.

Microorganisms inoculated in a culture medium can potentially undergo aerobic and/or anaerobic biochemical reactions. These biochemical reactions can produce acidic or basic byproducts, which in turn can lower or raise the pH of the culture medium and, where a pH indicator is used, cause a color change of the culture medium. The hydrophobic barrier substantially prevents acidic and/or basic byproducts potentially produced by a microorganism inoculated in one culture medium from affecting the pH of another culture medium. Thus, two culture media, in which a microorganism could respectively produce an acidic and basic byproduct, can be used in the same chamber 22 of the receptacle 14 because the acidic and basic byproducts produced by the microorganism in one culture medium will not substantially affect the pH of the other culture medium.

Although the apparatus 10 illustrated in FIG. 1 includes only two culture media and one hydrophobic barrier, the apparatus can potentially include more than two culture media and/or more than one hydrophobic barrier.

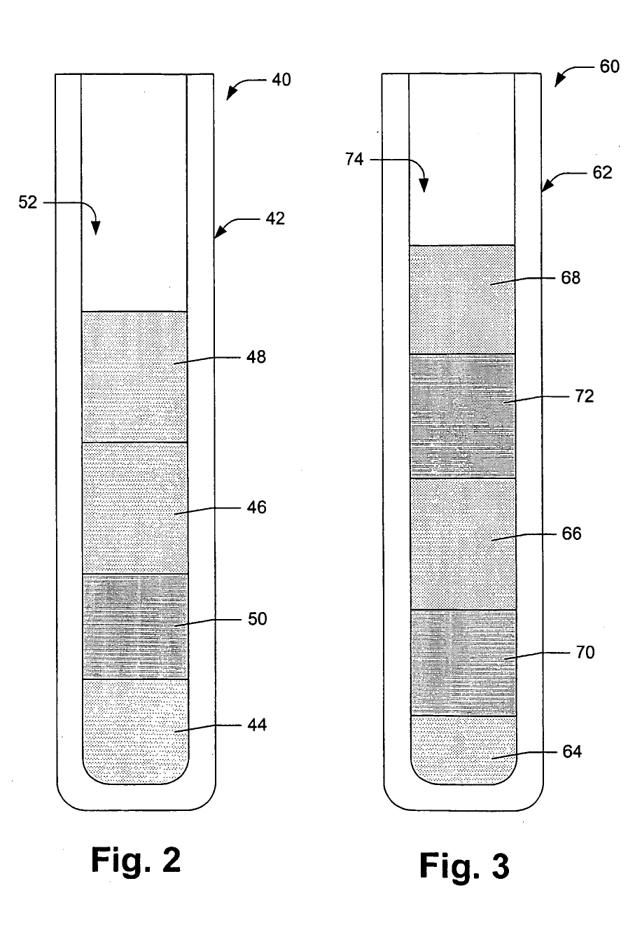

For example, FIG. 2 shows an apparatus 40 that includes a receptacle 42, which contains a first culture medium 44, a second culture medium 46, a third culture medium 48, and a hydrophobic barrier 50. The first culture medium 44, the second culture medium 46, the third culture medium 48, and the hydrophobic barrier 50 are provided as axially aligned layers within a chamber 52 of the receptacle 40. The hydrophobic barrier 50 substantially separates the first culture medium 44 and the second culture medium 46.

Additionally, FIG. 3 shows an apparatus 60 that includes a receptacle 62, which contains a first culture medium 64, a second culture medium 66, a third culture medium 68, a first hydrophobic barrier 70, and a second hydrophobic barrier 72. The first culture medium 64, the second culture medium 66, the third culture medium 68, the first hydrophobic barrier 70, and the second hydrophobic barrier 72 are provided as axially aligned layers within a chamber 74 of the receptacle 62. The first hydrophobic barrier 70 substantially separates the first culture medium 64 and the second culture medium 66, while the second hydrophobic barrier 72 substantially separates the second culture medium 66 and the third culture medium 68.

Other embodiments that include at least two culture media and at least one hydrophobic barrier that substantially separates the at least two culture media are within the scope of the present invention.

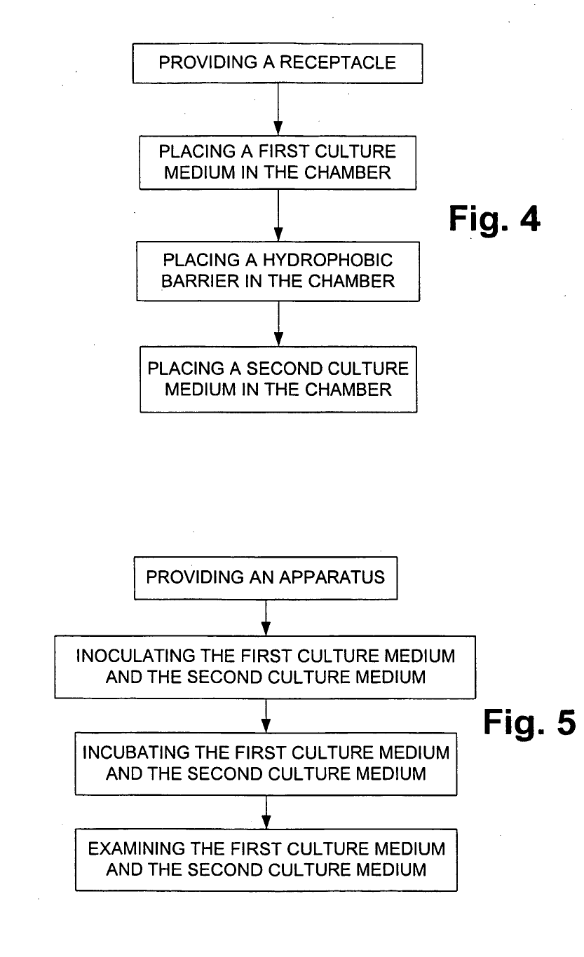

FIG. 4 is a schematic illustration of a method of preparing an apparatus that includes a receptacle, which contains a first culture medium, a second culture medium, and a hydrophobic barrier that substantially separates the first culture medium and the second culture medium.

In the method, a receptacle that includes an axially extending chamber is provided. A sample of the first culture medium is placed in the chamber. The sample of first culture medium can be in either solid or liquid form. The first culture medium can be used in solid form by mixing the first culture medium with a solidifying agent, such as an agar. The first culture medium placed in the chamber of the receptacle forms a first layer at the bottom of the chamber.

A layer of the hydrophobic material can then be placed within the chamber of the receptacle over the first culture medium. The layer of hydrophobic material should be axially aligned over the first culture medium and substantially cover the first culture medium.

A sample of a second culture medium can then be placed within the test tube over the layer of hydrophobic material. The second sample of culture medium, like the sample of the first culture medium, can be in liquid or a solid form and is, preferably, in solid form. The sample of second culture medium forms a layer that is axially aligned with the first culture medium and hydrophobic barrier and substantially covers the hydrophobic barrier.

A method of detecting a microorganism in accordance with an aspect of the present invention is illustrated schematically in FIG. 5. In the method, an apparatus in accordance with the apparatus of FIG. 1 is provided. The first culture medium and the second culture medium of the apparatus are then inoculated with a sample of a microorganism. The first culture medium and the second culture can be inoculated by placing a sample of the microorganism on the tip of a thin member, such as a metal wire or wooden stick. The thin member, with the sample of the microorganism, is stabbed through the second culture medium, the hydrophobic barrier, and the first culture medium. Stabbing the thin member through the second culture medium and the first culture medium causes at least some of the microorganism on the tip of the thin member to be transferred to the first and second culture media. Mixing may occur between the first culture medium and the second culture medium as a result of the stabbing, but this mixing is insignificant and does render the first culture medium and the second culture medium ineffective for their intended use.

The receptacle containing the inoculated first culture medium and second culture medium can then be incubated at a predetermined temperature and for a predetermined duration of time for the microorganism to potentially undergo a biochemical reaction with the first culture medium and the second culture medium. Following incubation, the first culture medium and the second culture medium are examined to determine if the microorganism reacts with the first culture medium and the second culture medium. Typically, where the culture medium includes a pH indicator, a biochemical reaction will be indicated by a visible change in color of the culture medium.

The following examples illustrate an apparatus that was prepared in accordance with one aspect of the present invention. The apparatus was a single tube screen that was used as a means for preliminary characterization as to whether Salmonella and/or Shigella bacteria were present in a stool sample of a patient suffering from a gastrointestinal disorder, such as very bloody diarrhea, enterocolitis, or gastritis.

### EXAMPLE 1

The single tube screen in accordance with one aspect of the present invention was provided. The single tube screen included a test tube that contained three differential culture media. The three differential culture media were used to screen isolates from stool cultures, which were suspected to represent Salmonella or Shigella bacteria species. The three differential culture media were provided in the test tube as three separate layers.

The single tube screen also included a hydrophobic barrier. The hydrophobic barrier substantially separated the first differential culture medium from the second differential culture medium within the test tube. The hydrophobic barrier consisted of a semi-solid hydrophobic material. The semi-solid hydrophobic material was Petrolatum, which was commercially available from Fischer Scientific.

The single tube screen was prepared by first providing a 20 mL test tube. 3.0 mL of the first differential culture medium was then placed within the test tube. The first differential culture medium consisted of a urea agar medium that was prepared by mixing at an elevated temperature 1 gram of yeast extract, 12 grams of BACTO Agar, and (10×) urea agar base. The urea agar base was reconstituted in accordance with the manufacturer's instructions (Difco). The pH of the urea agar medium was adjusted to 6.5. The urea agar medium upon being placed in the test tube cooled to room temperature (25° C.) and formed a light yellow semi-solid layer in the bottom of the test tube.

A 2.0 mL sample of the semi-solid hydrophobic material was then placed within the test tube. The sample of hydrophobic material, upon being placed in the test tube, formed a layer that substantially covered the layer of the urea agar medium.

4.0 mL of the second differential medium was then placed within the test tube. The second differential culture medium consisted of a lysine agar medium that was prepared by mixing at an elevated temperature 1 gram of dextrose (BACTO), 5 grams of peptone (BACTO), 3 grams of yeast extract, 5 grams of sodium chloride, 0.02 grams of brom-cresol purple, 10 grams of L-lysine, and 12 grams of BACTO agar. The pH of the lysine agar medium was adjusted to 6.5. The lysine agar medium, upon being placed within the test tube, cooled to room temperature (25° C.) and formed a semi-solid layer that covered the layer of hydrophobic material. The layer of lysine agar medium was substantially separated from the layer of urea agar medium by the layer of hydrophobic material.

2 ml of the third differential culture medium was then placed in the test tube. The third differential culture medium consisted of a lysine iron agar medium that was prepared by mixing, at an elevated temperature, 1 gram of dextrose (BACTO), 3 grams of yeast extract, 5 grams of salt, 0.02 grams of brom-cresol purple, 10 grams of L-lysine, 12 grams of BACTO agar, 5 grams of BACTO peptone, 0.3 grams of sodium thiosulfate, and 0.3 grams of ferrous sulfate. The test tube was tilted so that the lysine iron agar medium, upon cooling, formed a slant that covered the lysine agar medium.

Once prepared, the single tube screen was inoculated with an isolate of gram-negative bacteria obtained from a bacteria culture of a stool sample suspected of including Salmonella and/or Shigella. The isolate was obtained by incubating a sample of a patient's fecal matter and a differential culture medium in a petri dish, and then identifying and isolating the suspected bacteria. The differential culture medium used to culture the suspected bacteria in the fecal matter was a MacConkey culture medium. Hektoen Enteric culture medium has also been found to be effective for culturing the suspected bacteria.

The single tube screen was inoculated with the isolate of gram-negative bacteria by placing a specimen of the isolate of gram negative bacteria on the tip of a thin wooden stick. The thin wooden stick, with the isolate of bacteria, was stabbed through the lysine iron agar slant, the lysine iron agar layer, the Petrolatum layer, and the urea agar layer so that an amount of the bacteria was placed in each medium.

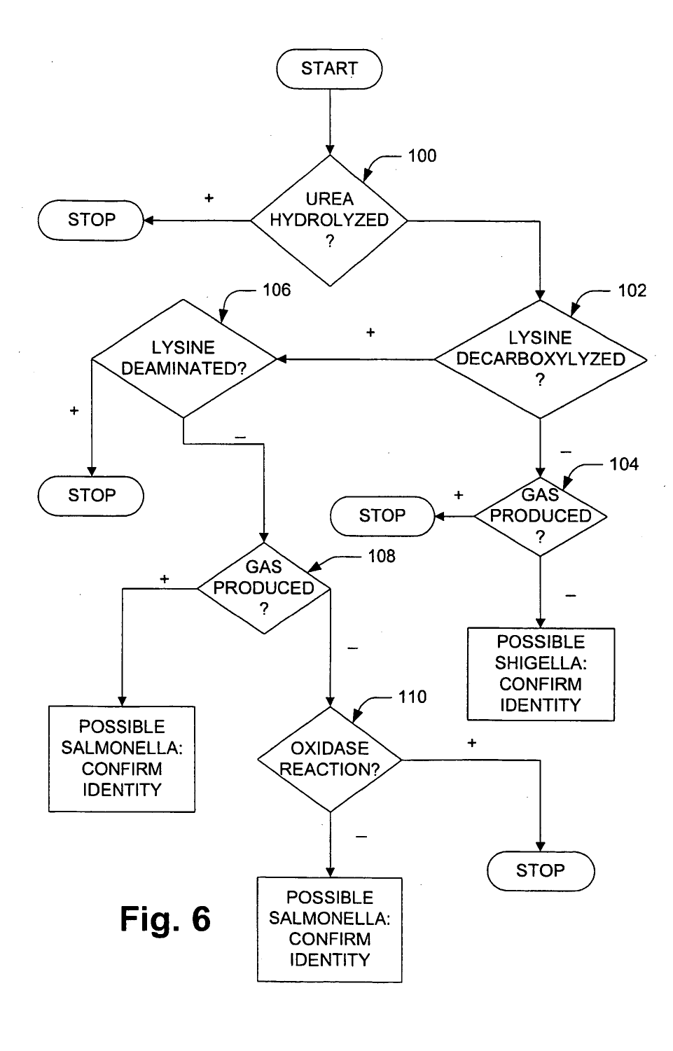

The single tube screen inoculated with the isolate of bacteria was incubated for about 18 to about 24 hours. Following incubation, the single tube screen was examined to determine if Salmonella and/or Shigella were present. Determination of whether Salmonella and/or Shigella was present was performed by following the process steps of the flow chart illustrated in FIG. 6.

Referring to FIG. 6, the first step 100 of the process was to determine whether the bacteria inoculated hydrolyze the urea in the urea agar medium. Bacteria that hydrolyze urea produce ammonia as a byproduct. Ammonia increases the pH of the urea agar medium, which in turn causes the urea agar medium to exhibit a color change from a pale yellow to pink or red.

If the urea agar medium had a pink or red color, the bacteria react (i.e., have a positive reaction) with the urea agar medium. Bacteria, which reacted with urea in the urea agar medium, were non-pathogens (i.e., not Salmonella and/or Shigella) and no further process steps were followed. If the urea agar medium had a yellow color, the bacteria did not react (i.e., had a negative reaction) with the urea in the urea agar medium.

Following a negative reaction with the urea, the process path was followed to 102 to determine if the isolated bacteria underwent a decarboxylation reaction with the lysine in the lysine agar medium. Decarboxylation of lysine occurs by an alkaline reaction, which causes the lysine agar medium to change color from yellow to purple. If the lysine agar medium had a purple color, the inoculated bacteria decarboxylized (i.e., had a positive reaction with) the lysine agar medium. If the lysine agar medium had a yellow color, the bacteria did not decarboxylize (i.e., had a negative reaction with) the lysine.

Following a negative decarboxylation reaction with the lysine in the lysine agar medium, the next step 104 in the process was to determine if gas was produced in the lysine agar medium. Gas is typically evidenced by cracks in the lysine agar medium. If gas was observed, the lysine agar medium was a non-pathogen and no further process steps were followed. If gas was not observed, the bacteria inoculated in the single tube screen was potentially Shigella and further analytical testing would be performed by Vitek, Microscan, and API20E methods to confirm the single tube screen results.

Following a positive decarboxylation reaction with the lysine in the lysine agar medium, the next step 106 was to determine if the lysine in the lysine iron agar slant was deaminated by the isolate of bacteria. Deamination is aerobic and occurs by an alkaline reaction, which causes the lysine iron agar slant to change color from yellow to purple-red. If a purple-red color was observed in the lysine iron agar slant, the bacteria deaminated (i.e., has a positive reaction with) the lysine. Bacteria that are capable of deaminating lysine are non-pathogens so no further process steps are followed. If the lysine iron agar slant did not have a purple-red color, the bacteria did not deaminate (i.e., had a negative reaction with) the lysine in the lysine iron agar slant.

Following a negative deamination reaction with the lysine in the lysine agar medium, the next step. 108 in the process was to determine if gas was observed in the lysine agar medium. The gas is typically evidenced by cracks in the lysine agar medium. If gas was observed, in the lysine agar medium, the bacteria inoculated in the single tube screen was potentially Salmonella, and further analytical testing would be performed by Vitek, Microscan, and API20E methods to confirm the single tube screen results.

If gas was not observed in the lysine agar medium, a separate isolate of bacteria from the stool sample was tested with an oxidase test 110. A typical oxidase test includes a solution of para-amino dimethlyanaline compound and an indicator. If the para-amino dimethlyanaline compound reacted with phenylalanine deaminase produced by the bacteria, a color change of the oxidase solution would be observed. If a positive reaction was observed, the bacteria was a non-pathogen. If a negative reaction was observed, that is no color change of the oxidase solution, the bacteria inoculated in the single tube screen was potentially Salmonella and further analytical testing would be performed by Vitek, Microscan, and API20E methods to confirm the single tube screen results. The oxidase test can be performed before the single tube screen to determine if a suspected bacteria is a non-pathogen or from the slant.

### EXAMPLE 2

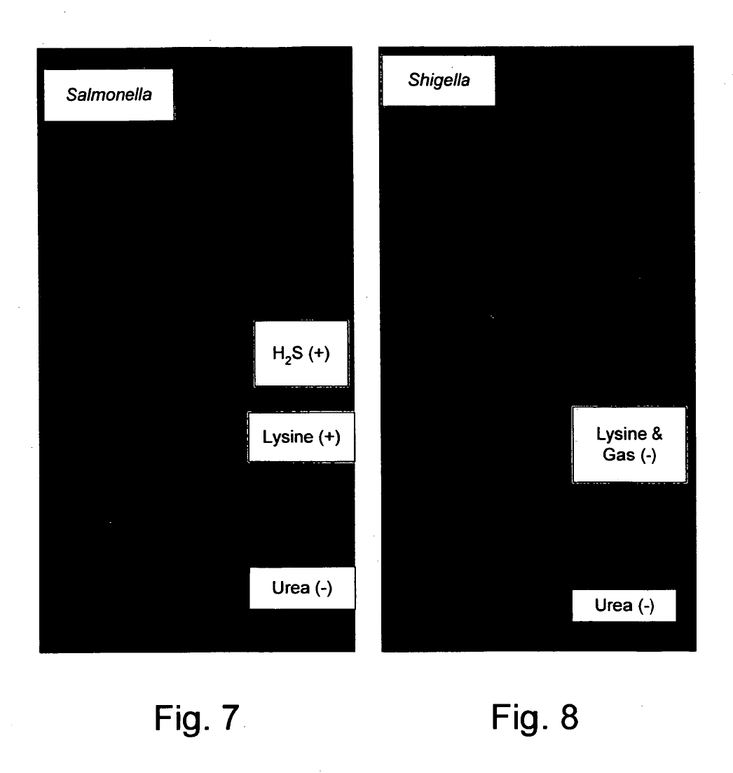

60 single tube screens were prepared in accordance with Example 1. Each of the single tube screens was inoculated with an isolate of Salmonella. The single tube screens were then incubated for 24 hours. FIG. 7 shows one of the incubated single tube screens inoculated with Salmonella. As can be seen in FIG. 7, the single tube screen exhibited a negative urea reaction, a positive lysine reaction, a negative deaminase reaction, and gas with hydrogen sulfide. The observed reaction results for Example 2 were in agreement with reaction results from traditional screening methods. Similar results were observed for the other single tube screens inoculated with Salmonella.

### EXAMPLE 3

30 single tube screens were prepared in accordance with Example 1. Each of the single tube screens was inoculated with an isolate of Shigella. The single tube screens were then incubated for 24 hours. FIG. 8 shows one of the incubated single tube screens inoculated with Shigella. As can be seen in FIG. 8, the single tube screen exhibited a negative urea reaction, a negative lysine reaction, and no gas. The observed reaction results for Example 3 were in agreement with reaction results from traditional screening methods. Similar results were observed for the other single tube screens inoculated with Shigella.

### EXAMPLE 4

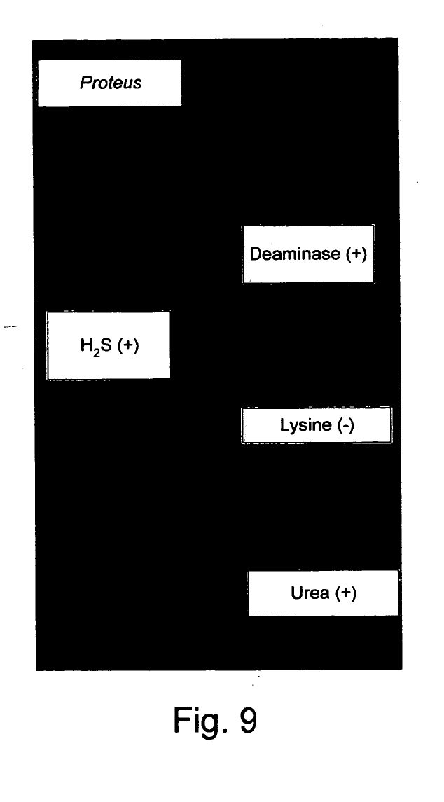

12 single tube screens were prepared in accordance with Example 1. Each of the single tube screens was inoculated with an isolate of Proteus. Proteus is a non-pathogen flora. The single tube screens were then incubated for 24 hours. FIG. 9 shows one of the incubated single tube screens inoculated with an isolate of Proteus. As can be seen in FIG. 9, the single tube screen exhibited a positive urea reaction, a negative lysine reaction, a positive deaminase reaction, and gas with hydrogen sulfide. The observed reaction results for Example 4 were in agreement with reaction results from traditional screening methods. Similar results were observed for the other single tube screens inoculated with Proteus.

### EXAMPLES 5-12

Additional single tube screens were prepared and inoculated with non-pathogen microorganisms. The microorganisms tested, the number of isolates per organism tested, and the accuracy of the test results compared with traditional screening methods are listed in the following Table.

TABLE

| | | | Percent agreement |
|---|---|---|---|
| | | # of Isolates | with traditional |
| EX | Organism | Tested | methods |
| 5 | Citrobacter | 10 | 90 (9/10 due to |
| | | | neg. urea) |
| 6 | Morganella | 6 | 83 (5/6 due to |
| | | | positive lysine) |
| 7 | Pseudomonas | 1 | 100 |
| 8 | Hafnia alvei | 2 | 100 |
| 9 | E. coli | 1 | 100 |
| 10 | Enterobacter | 1 | 90 (9/10 due to |
| | | | negative urea) |
| 11 | Klebsiella | 1 | 100 |
| | pneumoniae | | |
| 12 | Providencia | 4 | 100 |

Examples 2 and 3 indicate that the single tube screen is a useful method to identify Salmonella and Shigella as the single tube screen correctly identified 100% of the samples tested. With regard to the isolates of non-pathogens that were tested (Examples 4-9), the single tube screen results agreed with results from traditional screening methods 92% of the time (35/38). Compared to traditional screening methods, the single tube screen appeared to be slightly less sensitive for the detection of urea, slightly more sensitive for the detection of hydrogen sulfide production, and equivalent to the detection of lysine decarboxylation and gas production. It was also noted that a strong urea reaction could potentially produce a false-positive lysine reaction. Therefore, when evaluating the single tube screens after incubation it is essential that the process path of the flow chart be followed.

From the above description of the invention, those skilled in the art will perceive improvements, changes and modifications. Such improvements, changes and modifications within the skill of the art are intended to be covered by the appended claims.

Having described the invention, I claim:

1. An apparatus for the detection of a microorganism, said apparatus comprising: a chamber, a first culture medium disposed within said chamber, a second culture medium disposed within said chamber, and a barrier substantially separating said first culture medium and said second culture medium within said chamber, said barrier comprising a semi-solid, hydrophobic material.

2. The apparatus of claim 1 wherein said chamber is defined by a wall and said wall is transparent.

3. The apparatus of claim 1 wherein at least one of said first culture medium and said second culture medium is water based.

4. The apparatus of claim 3 wherein said first culture medium and said second culture medium have a different composition.

5. The apparatus of claim 1 wherein said first culture medium and said second culture medium are inoculated by passing a microorganism through said first culture medium, said second culture medium, and said hydrophobic barrier.

6. The apparatus of claim 1 wherein said first culture medium and said second culture medium are pH sensitive.

7. The apparatus of claim 6 wherein said hydrophobic barrier prevents a pH change in one of said first culture medium and said second culture from affecting the pH of said other of said first culture medium and said second culture medium.

8. The apparatus of claim 1 wherein said semi-solid hydrophobic material comprises petrolatum.

9. The apparatus of claim 1 wherein at least one of said first culture medium and said second culture medium are in semi-solid or substantially solid form.

10. The apparatus of claim 1 wherein the first culture medium, the second culture medium, and the hydrophobic are axially aligned within said chamber.

11. An apparatus for the detection of a microorganism, comprising: a chamber, at least two culture media disposed within said chamber, and a barrier that substantially separates said at least two culture media within said chamber, said barrier comprising a semi-solid, hydrophobic material.

12. The apparatus of claim 11 wherein a first culture medium, a second culture medium, and a third culture medium are disposed within said chamber, and said barrier substantially separates said first culture medium and said second culture medium.

13. The apparatus of claim 12 wherein said first culture medium, said second culture medium, said third culture medium, and said barrier are axially aligned within said chamber.

14. The apparatus of claim 13 wherein said first culture medium comprises a urea agar medium, said second culture medium comprises a lysine agar medium, said third culture medium comprises a lysine iron agar medium, and said barrier comprises petrolactum.

15. A method of detecting a microorganism, said method comprising the steps of:
providing an apparatus that includes a chamber, a first culture medium disposed within said chamber, a second culture medium disposed within said chamber, and a barrier, said barrier substantially separating said first culture medium and said second culture medium within said chamber, said barrier comprising a semi-solid, hydrophobic material;
inoculating said first culture medium and said second culture medium with a microorganism;
incubating said inoculated first culture medium and said inoculated second culture medium; and
examining said incubated first culture medium and said incubated second culture medium.

16. The method of claim 15 wherein said first culture medium and said second culture medium are inoculated by placing a sample of a microorganism on a thin member; and stabbing the thin member, with the microorganism, through the second culture medium, the barrier, and the first culture medium.

17. The method of claim 15 wherein said first culture medium comprises a urea agar medium, said second culture medium comprises a lysine agar medium, said third culture medium comprises a lysine iron agar medium, and said barrier comprises petrolactum.

18. The method of claim 17 wherein said microorganism is a bacterium.
